(19) United States
(12) Patent Application Publication
(10) Pub. No.: US 2022/0002795 A1
(43) Pub. Date: Jan. 6, 2022

(54) NANO-PEN SEQUENCING: AN INTEGRATED NANOTUBE AND TUNNEL GAP PLATFORM FOR POLYMER SEQUENCING

(71) Applicant: Wuhan University of Science and Technology, Wuhan City (CN)

(72) Inventors: Shuai Chang, Wuhan City (CN); Feng Liang, Wuhan City (CN); Huazhi Gu, Wuhan City (CN); Mingzhu Huang, Wuhan City (CN); Lei Yu, Wuhan City (CN)

(21) Appl. No.: 16/921,566

(22) Filed: Jul. 6, 2020

(51) Int. Cl. C12Q 1/6869; B01L 3/02; G01N 33/68; B82Y 15/00

(52) U.S. Cl. CPC C12Q 1/6869; B01L 3/021; G01N 33/6818; B01L 2200/12; B01L 2300/0645; B01L 2300/12; B01L 2300/16; B82Y 15/00

(57) ABSTRACT

The present invention provides methods, devices and systems for sequencing and/or analyzing a polymer and/or polymer unit. The polymer may include but not limited to DNA, RNA, a polysaccharide, or a protein. The device includes a nano-pen, which is a bifunctional nanopore/nanoelectrode, and a second electrode. The nano-pen electrode and the second electrode form a tunnel gap. Polymers passing through the nano-pen nanopore will be directed to the tunnel gap between electrodes. The electrodes are functionalized with a recognition reagent, and the reagent can transiently bind each polymer unit during its passage. When the transient bond forms, distinctive current signals are detected and recorded. The signals are utilized to analyze and identify the polymer and/or polymer unit.

### TECHNICAL FIELD

The present invention relates to methods, devices and systems for sequencing polymers, such as DNA, RNA, protein and polysaccharide.

### BACKGROUND TECHNOLOGY

Single nucleic acid bases of DNA, such as A, T, G, C can be detected and distinguished through recognition tunneling signals generated when single base unit is trapped in a tunnel gap between a pair of electrodes functionalized with recognition molecules. For example, PCT publication no. WO 2011/097171A1 (“Controlled tunnel gap device for sequencing polymers”) and U.S. publication no. 61/620,167 (“Electrodes for sensing chemical composition”), are all hereby incorporated by reference herein in their entireties. In spite of the single base resolution of the aforementioned recognition tunneling based method, a device and/or a method to direct each unit of a DNA polymer sequentially into the tunnel gap is a requisite to achieve the aim of sequencing polymers. Conventionally, an integration of a nanofabrication-derived nanopore with the tunnel gap has long been proposed, but it suffers from difficulties. For example, the nanofabrication process of a nanopore is always time and cost consuming. Second, aligning a nanometer-sized tunnel gap between a pair of electrodes with a nanometer-sized nanopore is almost prohibitive and difficult to batch produce in a reproducible manner. As a consequence, a device and/or a method for integration of tunnel gap with a conventional nanofabrication-derived nanopore show no industrial success to date.

In view of the foregoing, it would be desirable to provide improved methods, devices and systems for sequencing nucleic acid polymers. In one aspect according to some embodiments, methods, devices and systems for sequencing nucleic acid polymers are provided that employ a nanopore that is time and cost efficient in the fabrication process. In another aspect according to some embodiments, methods, devices and systems for sequencing nucleic acid polymers are provided that utilize a nanopore, that is simple to integrate with the tunnel gap between electrodes. One or both of these improvements and advantages, and/or other improvements and advantages can be provided with the present disclosure.

### SUMMARY OF THE INVENTION

Embodiments described herein provide devices, methods for sequencing polymers, such as DNA, or protein.

For example, some embodiments of the present disclosure provide devices, methods for sequencing polymers that utilize a nano-pen comprising an integrated nanotube and nanoelectrode, with the nanoelectrode (i) functionalized with one or more recognition molecules and (ii) capable for use to detect one or more chemical compositions of the polymer.

In some embodiments, a method for identifying a chemical composition and/or sequencing a polymer, and a corresponding device used in the method are provided. The device includes an integration of a first electrode and a nanotube. A second electrode is separated from the first electrode by about 1 to 4 nm. The first electrode, second electrode, or both have at least one recognition molecule chemically attached. In some embodiments, the recognition molecule comprises 4(5)-(2-mercaptoethyl)-1H imidazole-2-carboxamide.

In an embodiment, an apparatus and corresponding method for sensing a chemical composition and/or sequencing a polymer are provided. For example, in some embodiments, a polymer unit, such as a nucleic acid or an amino acid, is caused to pass through the nanotube and directed to the tunnel gap between electrically-separated electrodes, where at least one of the electrically-separated electrodes is functionalized with a recognition molecule. A polymer unit is recognized based on a current signal arising from the polymer unit passing through the tunnel gap. In some embodiments, the recognition molecule comprises 4(5)-(2-mercaptoethyl)-1H imidazole-2-carboxamide.

In some embodiments, a device for translocating one or more polymers is provided and comprises a nanotube and a first electrode.

In some embodiments, a second electrode is electrically separated from the first electrode by about 1 to 4 nm, at least one recognition molecule functionalized on the first electrode, and ate least one recognition molecule functionalized on the second electrode.

In some embodiments, at least one recognition molecule attached to the first electrode, at least one recognition molecule attached to the second electrode, or both comprise 4(5)-(2-mercaptoethyl)-1H imidazole-2-carboxamide.

In some embodiments, the electrodes are biased with respect to a reference electrode. In some embodiments, the bias of the electrode is maintained at between about +0.5V and −0.5V versus Ag/AgCl.

In some embodiments, an apparatus for identifying a chemical composition and/or sequencing a polymer is provided and may comprise means for causing a polymer to flow through a nanotube. Such embodiments may also include means for causing a polymer unit to pass through a tunnel gap between electrically separated electrodes, where at least one of electrodes is functionalized with a recognition molecule. Such embodiments may also include means for identifying a type of the polymer unit based on the current signals generated from the unit passing through the tunnel gap. Such means may be a computer processor performing data analysis to determine the type of the unit.

In some embodiments, a method of fabricating a device capable of translocating a polymer is provided and may comprise one or more of the following steps (and in some embodiments, a plurality, and in some embodiments, all steps); providing a dual-barrel nanopipette, filling one barrel with carbon and/or other conducting materials to form an electrode, chemically tethering at least one recognition molecule to the electrode.

In some embodiments, a method of fabricating a device capable of translocating a polymer is provided and may comprise one or more of the following steps (and in some embodiments, a plurality, and in some embodiments, all steps); providing a single-barrel nanopipette, depositing a thin layer of gold, or palladium, or other conducting materials to form an electrode at exterior of the nanopipette, chemically tethering at least one recognition molecule to the electrode.

In some embodiments, a method of detecting and sequencing a polymer is provided and may comprise one or more of the following steps (and in some embodiments, a plurality, and in some embodiments, all steps): providing a nano-pen consisting of an integrated nanotube and a first electrode, providing a second electrode separated from the first electrode by a gap distance of about 1 to 4 nm, functionalizing at least one recognition molecule to the first electrode, functionalizing at least one recognition molecule to the second electrode, causing a polymer to flow through the nanotube, directing a polymer unit to pass through the tunnel gap between electrically separated electrodes, identifying a type of polymer unit based on current signals generated during the passage of a unit through the tunnel gap. Such identifying may comprise using computer, processors, and the like, to perform data analysis to recognize a signature signal for a type of polymer unit so as to determine the polymer sequence.

### BRIEF DESCRIPTION OF THE FIGURES

The accompanying drawings show aspects that are incorporated in and constitute a part of the present invention. The descriptions herein explain some of the principles relating to the disclosed embodiments illustrated in the drawings.

FIG. 1 shows a schematic diagram of a nano-pen sequencing platform comprising an integrated nanotube with a tunnel gap created using a self-built scanning tunneling microscope according to some embodiments of the present disclosure;

FIG. 2 shows a zoomed-in view of the tunnel gap region in FIG. 1 showing how the recognition molecules are connected to the two electrodes and form hydrogen bonds with each individual unit of a DNA molecule translocating through the nanotube;

FIG. 3A shows a device according to some embodiments of the present disclosure fabricated by, for example, pulling a dual-barrel quartz pipette with a laser puller or other fabrication method. The fabricated dual-barrel nanopipette is then filled with carbon or other conductive materials inside one of the two barrels, keeping the other barrel hollow as a transmission pipeline;

FIG. 3B shows a device according to some embodiments of the present disclosure fabricated by, for example, pulling a single-barrel quartz pipette with a laser puller or other fabrication method. The fabricated nanopipette is then deposited with a thin layer of chromium and gold to make its exterior conductive, keeping the barrel hollow as a transmission pipeline;

FIG. 4A is a scanning electron microscope (“SEM”) image of an integrated nanotube/nanoelectrode fabricated from a dual-barrel nanopipette according to some embodiments of the present disclosure;

FIG. 4B is a scanning electron microscope (“SEM”) image of an integrated nanotube/nanoelectrode fabricated from a single-barrel nanopipette according to some embodiments of the present disclosure;

FIG. 5 is a photo image of the exemplary set up according to some embodiments of the present disclosure;

FIG. 6 illustrates the recognition molecule used to functionalize the electrodes according to some embodiments of the present disclosure;

FIG. 7 is a typical trace of tunnel current versus time for control background taken in 1 milli-Molar (mM) phosphate buffer using Au electrodes functionalized with 4(5)-(2-mercaptoethyl)-1H imidazole-2-carboxamide, according to some embodiments of the present disclosure. There is no detectable signal shown on the background;

FIG. 8 shows typical signal traces for four nucleotides and one methylated nucleotide when they are added to the tunnel junction according to some embodiments of the present disclosure. 100 μM in 1 mM phosphate buffer was used with the Au electrodes functionalized with 4(5)-(2-mercaptoethyl)-1H imidazole-2-carboxamide;

### DETAILED DESCRIPTION OF THE INVENTION

The principles and characteristics of the present invention are described on the basis of these drawings; the examples cited are provided only to interpret the present invention, not to limit its scope.

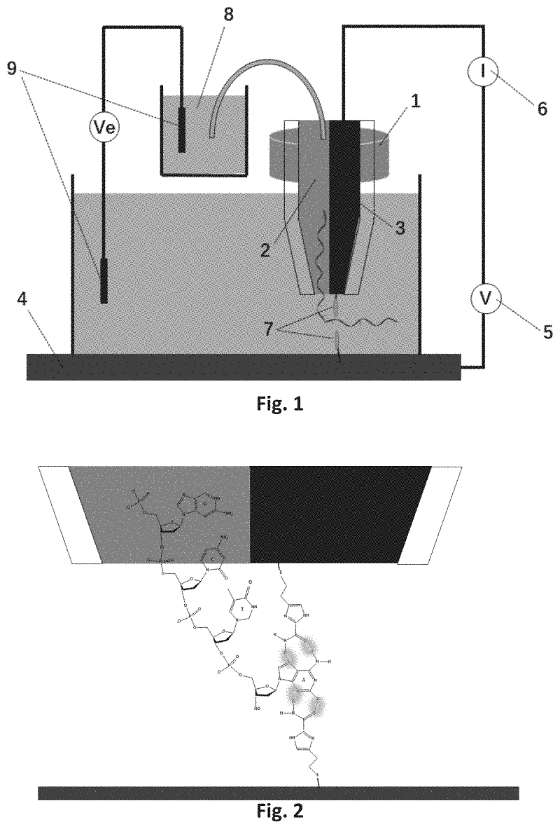

FIG. 1-2 show illustrative embodiments of a nano-pen sequencing platform, which is a polymer detecting and sequencing platform according to some embodiments of the present disclosure. FIG. 1 is representative of some embodiments based on a home-built scanning tunneling microscope platform. A piezoelectric positioner (1) holds a nano-pen consisting of a nanotube (2) and a nanoelectrode (3) at a distance (d) from a substrate electrode (4). In some embodiment, the electrode is gold, palladium, or other metal materials, including metal alloy. In some embodiments, the distance, d is set to about 2.5 nm using positioner (1). In some embodiments, the entire arrangement of nanotube (2), nanoelectrode (3) and substrate electrode (4) may be immersed in an aqueous solution.

Still referring to FIG. 1, in some embodiments, the polymer is directed into the tunnel junction by electrophoretic driving through the nano-pen nanotube (2). The aqueous electrolyte could be 1 to 100 mM phosphate buffer solution with a pH 7.0, or other working aqueous electrolytes. A voltage bias V (5) is applied between nanoelectrode (3) and substrate electrode (4), and the current I, though the junction is measured with a current preamplifier (6). Specifically, the electrodes are modified with one or more recognition molecules (7) that are strongly bonded to the metal electrodes, for example, via thiol anchor groups, and these recognition molecules facilitate non-covalent bonds with each individual polymer units. In one embodiment, the recognition molecule (7) attached to the first nanoelectrode (3) and/or second electrode (4) is 4(5)-(2-mercaptoethyl)-1H imidazole-2-carboxamide. Alternatively or in addition, other choice of recognition molecules may be attached to the electrodes. Polymer units, such as nucleosides of DNA or amino acids of protein passing through the tunnel gap will give rise to distinctive current spike signals that can be recorded and analyzed to recognize the unit in the tunnel gap and determine the polymer sequence by lining up the unit signatures.

Still referring to FIG. 1, in some embodiments, a reservoir (8) (shown in cross section) is connected to the open channel of nanotube (2) of a nano-pen. A polymer to be sequenced, such as DNA, is present in the fluid reservoir (8). The fluid may flow through the nanotube (2) or may be driven through the nanotube by an electrophoretic bias Ve applied between reservoir (8) and the aqueous solution using reference electrodes 9.

Exemplary illustration of nano-pen sequencing is shown in FIG. 2. A recognition molecule attached to the nanoelectrode of a nano-pen and a second recognition molecule attached to the substrate electrode capture a DNA nucleotide from a single strand DNA molecule which translocate through the nanotube of a nano-pen by electrophoresis.

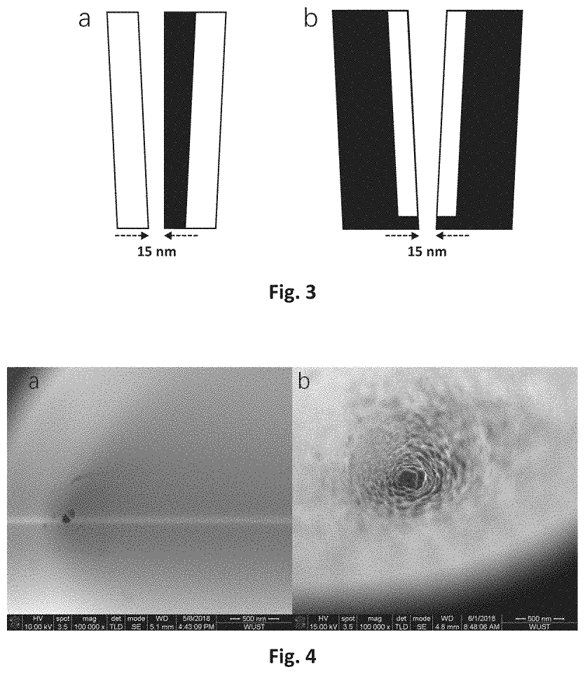

FIGS. 3A and 3B show two different types of nano-pens for polymer translocation and sequencing. FIG. 3A is a schematic layout of a type 1 nano-pen device according to some embodiments of the present disclosure. A cleaned quartz theta pipette is pulled using a laser pipette puller with the following parameters: HEAT=825, FIL=3, VEL=40, DEL=220, PUL=190 to create a dual nanopore nanopipette. One of the barrels of the dual-barrel nanopipette is filled by conductive material, such as carbon, gold or palladium, and the other barrel is kept hollow to translocate molecular solutions. In some embodiments, the diameter of the open pore of the nanotube is around 15 nm.

FIG. 3B is a schematic layout of a type 2 nano-pen device according to some embodiments of the present disclosure. A cleaned quartz pipette is pulled using a laser pipette puller with the following parameters: HEAT=825, FIL=3, VEL=40, DEL=220, PUL=190 to create a nanopipette. The fabricated nanopipette is then sequentially deposited with 10 nm chromium and 100 nm gold, for example, via thermal evaporator. In some embodiments, the diameter of the open pore of the nanotube is around 15 nm.

FIGS. 4A and 4B are SEM images of the nano-pen devices fabricated as described above. These images demonstrate that the fabrication of a nano-pen capable of molecular translocation through nanotube 2 and molecular detection via nanoelectrode 3 has been achieved.

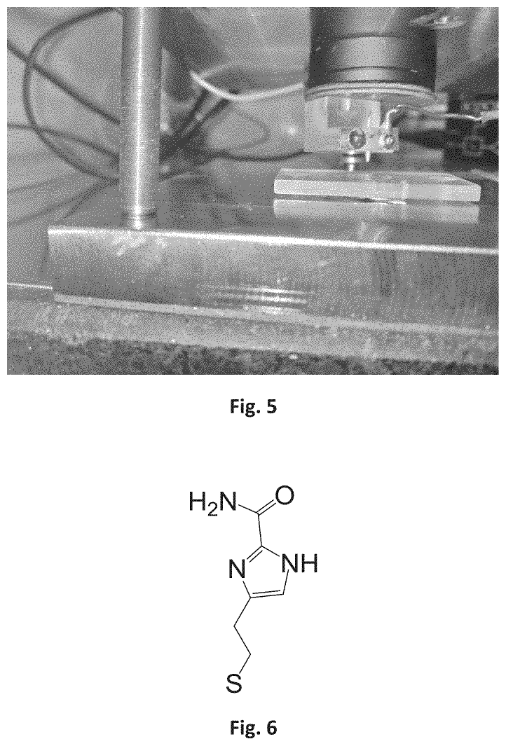

FIG. 5 shows an exemplary set up with a self-built scanning tunneling microscope according to some embodiments of the present disclosure. The device configurations described above in FIG. 1-5 are only illustrative. Any other suitable configurations of a device for sensing and sequencing a polymer may be used, including with respect to device geometry (e.g., length, thickness, width and positioning of the nanotube and nanoelectrode), materials selected for the nano-pen structure and/or the metal(s).

FIG. 6 shows the chemical structure of the recognition molecule. In various embodiments of the present disclosure, any suitable recognition molecule(s) can be attached to the first and/or second electrodes of a device for trapping and reading each individual units of a polymer through recognition tunneling signals. In some embodiments, the recognition molecule is 4(5)-(2-mercaptoethyl)-1H imidazole-2-carboxamide.

Nano-pen sequencing measurements were carried out using the recognition molecule described in connection with FIG. 6. In each instance, both gold substrate and a type 2 gold coated nano-pen were used. Newly fabricated nano-pen was rinsed with ethanol; the gold substrates were annealed with hydrogen flame. A nano-pen device and a gold substrate were immersed in a 1 mM solution of recognition molecule for overnight, then rinsed thoroughly with ethanol and blow dry with nitrogen. Measurements were carried out with a home built STM controlled with self-made Labview program. The program records current versus time data from where the amplitude and other features of the jump current signals are measured. 1 mM PBS buffer solution (pH=7.4) was filled in reservoir 8 and 9 for control measurements, and 10 μM solution of nucleoside monophosphates were added to reservoir 8 for recognition measurements. Before taking the data, the system was stabilized for at least 1 hour without adding molecular solution or applying any bias between nanoelectrode 3 and substrate 4. After the system was stabilized, a substrate bias of 0.1 V was applied and different setpoint current was set, then the recognition tunneling signal was recorded.

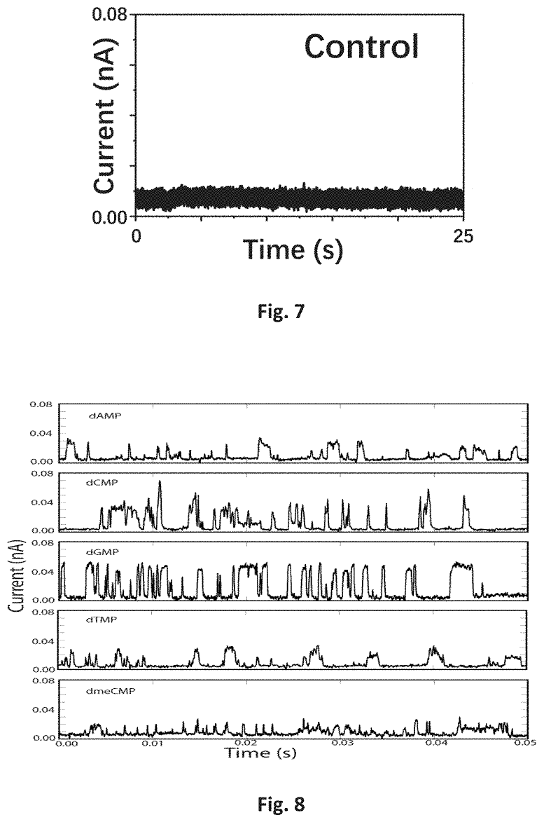

FIG. 7 shows the control measurements without adding nucleoside molecules. The tunneling current was measured at a setpoint of 4 pA using a gold coated nano-pen and gold substrate functionalized with recognition molecule 4(5)-(2-mercaptoethyl)-1H imidazole-2-carboxamide.

FIG. 8A-E show recognition tunneling measurements with A, C, G, T and methylated C nucleoside monophosphates added in reservoir 8 and driven by electrophoresis under reference electrodes. The tunneling current was measured at a setpoint of 4 pA using a gold coated nano-pen and gold substrate functionalized with recognition molecule 4(5)-(2-mercaptoethyl)-1H imidazole-2-carboxamide.

Any and all references to publications or other documents, including but not limited to, patents, patent applications, articles, webpages, books, etc., presented in the present application, are herein incorporated by reference in their entirety.

Although a few variations have been described in detail above, other modifications are possible. For example, any logic flow depicted in the accompanying figures and described herein does not require the particular order shown, or sequential order, to achieve desirable results. Other implementations may be within the scope of at least some of the claims present in this disclosure.

Example embodiments of the devices, systems and methods have been described herein. As noted elsewhere, these embodiments have been described for illustrative purposes only and are not limiting. Other embodiments are possible and are covered by the disclosure, which will be apparent from the instructions contained herein. Thus, the breadth and scope of the disclosure should not be limited by any of the above described embodiments but should be defined only in accordance with claims supported by the present disclosure and their equivalents. Moreover, embodiments of the subject disclosure may include methods, systems and devices which may further include any and all elements from any other disclosed methods, systems, and devices, including any and all elements corresponding to methods, systems and devices for sensing chemical composition and sequencing polymers, such as DNA or protein. In other words, elements from one or another disclosed embodiment may be interchangeable with elements from other disclosed embodiments. In addition, one or more features/elements of disclosed embodiments may be removed and still result in patentable subject matter (and thus, resulting in yet more embodiments of the subject disclosure)

1. A device for detecting and sequencing a polymer, comprising:
a nano-pen consisting of a combination of a nanotube and an electrode;
a second electrode that forms a tunnel gap with the nano-pen electrode;
a first recognition molecule tethered to the nano-pen electrode;
a second recognition molecule tethered to the second electrode;
wherein the first and second recognition molecules can each form a transient bond with the polymer unit and detectable signal is recorded when bond forms.

2. The device of claim 1 wherein the nano-pen is made from a theta quartz pipette that is pulled with a laser puller to produce a dual-barrel nanopipette. One barrel is made into an electrode through electrode material filling, while the other barrel serves as a transmission pipeline.

3. The device of claim 1 wherein the nano-pen is made from a single-barrel quartz pipette that is pulled with a laser puller to produce a nanopipette. It is subsequently coated with 10 nm chromium and 100 nm gold to make its exterior a conductive electrode and interior an open nanotube.

4. The device of claim 1, wherein the first and/or second electrode comprises gold, palladium or alloy metals.

5. The device of claim 1, wherein the at least one recognition molecule tethered to the nano-pen electrode, the at least one recognition molecule tethered to the second electrode, or both comprise 4(5)-(2-mercaptoethyl)-1H imidazole-2-carboxamide.

6. The device of claim 1, wherein the recognition molecule and the tunnel gap are configured to produce distinctive signal for each polymer unit when first and second recognition molecules transiently bond to each individual unit of the polymer.

7. The device of claim 1, wherein the tunnel gap is sized about 1 to 4 nm.

8. The device of claim 1, wherein the transient bond is a noncovalent bond, including but not limited to hydrogen bonds, ionic-dipole interaction, hydrophobic interaction.

9. The device of claim 1, wherein the polymer is DNA or RNA and the unit is a nucleotide.

10. The device of claim 1, wherein the polymer is a protein and the unit is an amino acid.

11. The device of claim 1, wherein the polymer is a carbohydrate and the unit is a monosaccharide or a sugar.

12. The device of claim 1, further comprising:
a first fluid reservoir connecting to the open channel of nano-pen via a transmission pipe;
a second fluid reservoir immersing the nano-pen and the second electrode;
said first and second fluid reservoirs are separated by at least one nanopore through which the polymer may flow through.

13. The device of claim 12, further comprising a first driving electrode in said first reservoir and a second driving electrode in said second reservoir to apply a bias.

14. An apparatus for sensing and sequencing the chemical composition of a polymer, comprising:
means for driving a polymer unit, such as a nucleic acid base or an amino acid to translocate through a nano-pen nanotube and pass through a tunnel gap electrically separated between a nano-pen nanoelectrode and a second electrode, wherein at least one of the electrically separated electrodes is functionalized with a recognition molecule; and
means for identifying a type of the polymer unit based on the tunneling current signals arising from the polymer unit passing through the tunnel gap.

15. The apparatus of claim 14, wherein the recognition molecule comprises: 4(5)-(2-mercaptoethyl)-1H imidazole-2-carboxamide.

16. A method of fabricating a device capable of sequencing a polymer, comprising:
providing a dual-barrel nano-pen combining a first electrode and a nanotube;
providing a second electrode separated from the first electrode with a distance of about 1 to 4 nm;
functionalizing at least one recognition molecule to the first electrode;
functionalizing at least one recognition molecule to the second electrode.

17. The method of claim 16, wherein the nano-pen is fabricated by pulling a dual-barrel pipette with a laser puller to create a dual-barrel nanopipette.

18. The method according to claim 16, wherein the first barrel is filled with carbon to make it a solid carbon nanoelectrode. Exposed region of said carbon nanoelectrode is coated with gold or palladium.

19. The method according to claim 16, wherein the second barrel serves as a nanotube for polymer to flow through.

20. A method of fabricating a device capable of sequencing a polymer, comprising:
providing a single-barrel nano-pen combining a first electrode and a nanotube;
providing a second electrode separated from the first electrode with a distance of about 1 to 4 nm;
functionalizing at least one recognition molecule to the first electrode;
functionalizing at least one recognition molecule to the second electrode.

21. The method of claim 20, wherein the nano-pen is made by pulling a single-barrel pipette with a laser puller to produce a nanopipette. It is subsequently deposited with 10 nm Cr and 100 nm gold to make it a conductive electrode at exterior of the nano-pen, combined with an internal nanotube.

22. A method of detecting and/or sequencing a polymer, said method comprising:
a) allowing a polymer unit flow into a tunnel gap formed between a first electrode functionalized with a first recognition molecule and a second electrode functionalized with a second recognition molecule;
b) generating transient noncovalent bond between said polymer unit and first and second recognition molecule;
c) recording a distinctive detectable signal when bond forms; and
d) repeating a)-c) for each successive unit of said polymer.
